(19) United States
(12) Patent Application Publication
(10) Pub. No.: US 2007/0156145 A1
(43) Pub. Date: Jul. 5, 2007

(54) Therapeutic constructions, spinal plates, cervical plates, hooks and screws

(75) Inventors: John J. Demakas, Spokane, WA (US); Brent W. Johnston, Spokane, WA (US)

Correspondence Address: WELLS ST. JOHN P.S., 601 W. FIRST AVENUE, SUITE 1300, SPOKANE, WA, 99201, US

(73) Assignee: Kentomia, LLC

(21) Appl. No.: 11/322,821

(22) Filed: Dec. 30, 2005

(51) Int. Cl. A61F 2/30

(52) U.S. Cl. 606/69

(57) ABSTRACT

The invention includes skeletal support structures. The structures can be spinal plates (such as cervical plates), rods, hooks, vertebral spacers, vertebral structural replacement, or screws; and can be formed of aromatic polyamide material (such as para-aramid). The screws and/or hooks can contain pores configured to receive growing bone to enhance union of the screws and/or hooks with skeletal material. The invention also includes therapeutic constructions containing non-metallic structures attached to vertebrae through non-metallic fasteners.

### TECHNICAL FIELD

The invention pertains to therapeutic constructions, spinal plates, cervical plates, hooks and screws. In some aspects, structures of the present invention can be non-metallic and/or porous.

### BACKGROUND OF THE INVENTION

Numerous structures have been developed for therapeutic attachment to skeletal regions. Such structures can include, for example, various screws, hooks, plates, pins, cages and rods. Therapeutic uses of such structures can include, for example, temporary support to mobilize a skeletal region during healing in response to injury (for instance, screws, hooks, rods and/or plates utilized to mobilize a fractured bone during healing of the fracture), permanent support to replace a skeletal segment (for example, a knee or hip replacement), or permanent support to provide additional support beyond that offered by a skeleton region compromised by injury, disease, aging or genetic defect (for example, spinal plates, cages, hooks and rods provided for additional support beyond that offered by a deteriorated spine).

Ideally, structures utilized for attachment to skeletal regions will be formed of compositions having high strength per unit volume and per unit weight, so that the structures can be kept small and light while still providing sufficient support. Thus, metal alloys have traditionally been utilized for such structures. However, metal alloys create their own problems when utilized as support structures. For instance, the metal alloys will scatter radiation during diagnostic exams of adjacent skeletal regions, which complicates examination of the skeletal regions. Further, metal alloys utilized for medical treatment cannot be distinguished from metals utilized in weapons during routine screening (such as, for example, airport security screening) which can lead to embarrassment and delay for persons having metal alloy support structures.

Also, the metal alloys do not enable some devices to be formed to small enough size to avoid some undesired complications. For instance, cervical plates are frequently placed between the spine and the esophagus within the neck of a patient. Frequently, the cervical plates are thick enough that the patient is aware of the plate during swallowing due to some interference of the plate with the esophagus. It is desired to create medical devices which are small enough that patients are completely unaware of the devices after the devices are in place. Presently, cervical plates are typically at least 1.5 millimeters (mm) thick, and it is desired to develop cervical plates which can be thinner while still providing sufficient support.

### SUMMARY OF THE INVENTION

In one aspect, the invention includes a spinal plate comprising aromatic polyamide material. The spinal plate can be a cervical plate in some aspects of the invention, and in particular aspects of the invention the aromatic polyamide material can be para-aramid (for instance, Kevlar™).

In one aspect, the invention includes a cervical plate that is less than or equal to about 1.5 mm thick; and that comprises, consists essentially of, or consists of a composite of aromatic polyamide material and carbon.

In one aspect, the invention includes a therapeutic construction. The construction comprises a segment of a spinal column containing a pair of vertebrae and a disk between the vertebrae. The construction also comprises a non-metallic structure attached to each vertebra of the pair of vertebrae with non-metallic fasteners. In some aspects, the non-metallic fasteners can be screws, and in particular aspects such screws can have pores extending therein.

In one aspect, the invention includes a screw configured to directly engage a bone. The screw comprises a shaft that is at least partially threaded, and comprises at least one pore extending into the shaft and configured to receive growing bone structure to enhance union of the screw with bone. In particular aspects, the screw can be of a composition comprising, consisting essentially of, or consisting of aromatic polyamide material.

In one aspect, the invention includes a hook configured to engage a bone. The hook can be porous and/or can be of a composition comprising, consisting essentially of, or consisting of non-metallic material (such as, for example, aromatic polyamide material).

### BRIEF DESCRIPTION OF THE DRAWINGS

Preferred embodiments of the invention are described below with reference to the following accompanying drawings.

FIG. 1 is a diagrammatic, top view of an exemplary spinal plate in accordance with an aspect of the present invention.

FIG. 2 is a diagrammatic, cross-sectional view along the line 2-2 of FIG. 1.

FIG. 3 is a diagrammatic, fragmentary view of an assembly comprising the plate of FIG. 1 attached to a pair of segments of a spinal column.

FIG. 4 is a diagrammatic view of an exemplary screw in accordance with an aspect of the present invention.

FIG. 5 is a diagrammatic view of another exemplary screw in accordance with an aspect of the present invention.

FIG. 6 is a diagrammatic view of an assembly comprising a spine and implant constructions attached to the spine, in accordance with an aspect of the present invention.

FIG. 7 is a cross-section along the line 7-7 of FIG. 6.

FIG. 8 is a diagrammatic side view of a disassembled pedicle screw assembly, in accordance with an aspect of the present invention.

FIG. 9 is a diagrammatic side view of a pedicle screw in accordance with another exemplary aspect of the present invention.

FIG. 10 is a cross-sectional side view of the pedicle screw of FIG. 9, and specifically is a view along the line 10-10 of FIG. 9.

FIG. 11 is a diagrammatic, cross-sectional side view of another embodiment of a screw formed in accordance with an aspect of the present invention.

FIG. 12 is a diagrammatic, cross-sectional side view of a skeletal region having an exemplary screw of the present invention retained therein.

FIGS. 13-16 show various exemplary hooks that can be formed in accordance with aspects of the present invention.

### DETAILED DESCRIPTION OF THE PREFERRED EMBODIMENTS

This disclosure of the invention is submitted in furtherance of the constitutional purposes of the U.S. Patent Laws “to promote the progress of science and useful arts” (Article 1, Section 8).

The invention includes structures that can be utilized to provide support to skeletal regions. The structures can be utilized in veterinary applications for treating animals, or can be utilized for treating humans. In particular aspects, the invention includes non-metallic spinal plates, such as, for example, cervical plates. In other particular aspects, the invention includes non-metallic screws that can be inserted into bone. The screws can contain pores therein, with the pores being configured so that bone structure grows into the pores to improve union of the screws with bone. The bone structure growth into the pores can be enhanced by providing one or more bone-growth-stimulating compositions within the pores. Additionally, or alternatively, bone cement can be provided within the pores. In yet other aspects, the invention includes non-metallic and/or porous hooks that can attach to skeletal structures.

The various non-metallic structures of the present invention can be formed from aromatic polyamide materials, such as, for example, aramids. Exemplary aramids are meta-aramids and para-aramids, with a well-known para-aramid being Kevlar™. The aromatic polyamide materials can be utilized alone, or in combination with carbon. Accordingly, non-metallic structures of the present invention can comprise, consist essentially of, or consist of aromatic polyamide materials; or non-metallic structures of the present invention can comprise, consist essentially of, or consist of composites of aromatic polyamide materials and carbon.

It is known in the art that aromatic polyamide materials can have high strength-to-weight ratio, and accordingly such materials are utilized in watercraft, bulletproof vests, and numerous other applications in which strength is desired and lightweight properties are also desired. Further, it is known in the art that polyamide/carbon composite materials can have particularly good strength-to-weight ratio. Accordingly, aromatic polyamide materials and polyamide/carbon composite materials are readily commercially available.

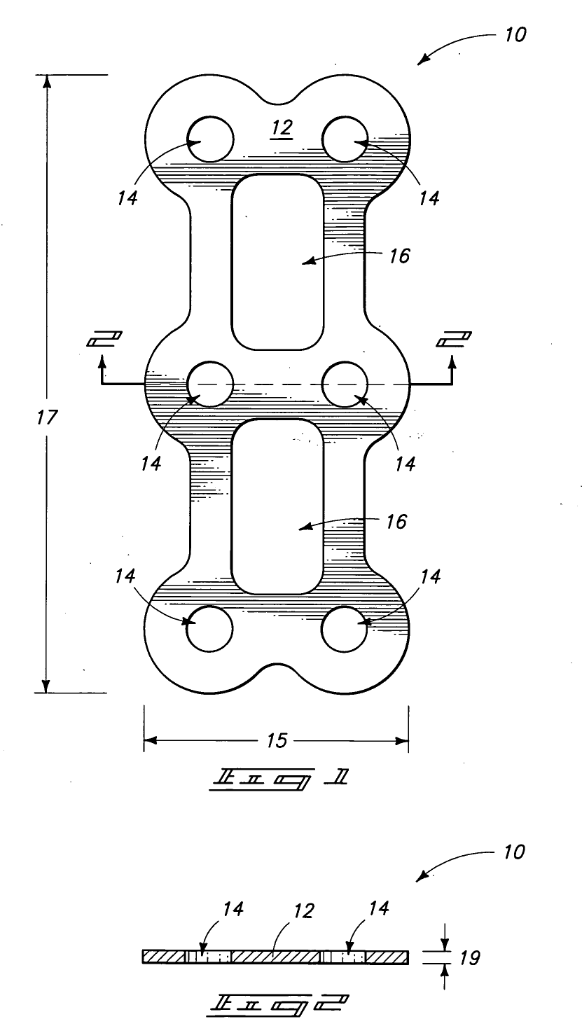

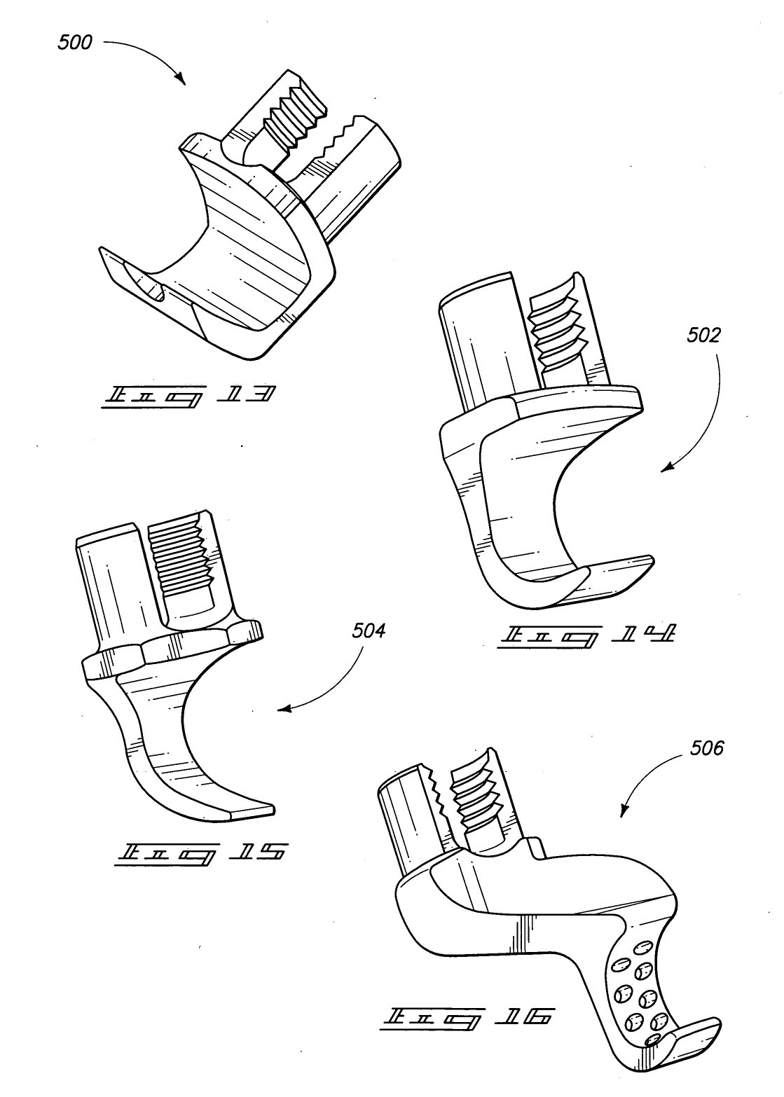

In some aspects, the present invention includes a recognition that the exceptional strength-to-weight properties of aromatic polyamide materials and polyamide/carbon composite materials can be of particular advantage for utilization in screws, plates, hooks and other structures utilized for supporting, or replacing, skeletal regions. Exemplary aspects of the invention are described below with reference to FIGS. 1-16.

Referring initially to FIGS. 1 and 2, such show an exemplary spinal plate 10 in accordance with an aspect of the present invention. Plate 10 comprises a structural material 12 having a plurality of holes 14 extending therethrough. The holes are configured for receiving screws, or other fasteners, ultimately utilized for retaining the plate to a spinal region. Additionally, a couple of windows 16 extend through the material 12, with such windows being configured to enable viewing of a bone graft provided within a spinal region behind plate 10. Persons of ordinary skill in the art will recognize that such windows are optional.

The shown plate is but one example of the numerous plates that can be utilized for attachment to spinal regions. It is to be understood that aspects of the present invention can be used for any spinal plate currently available, or which becomes available in the future. The spinal plate can be a cervical plate (in other words, can be configured for attachment to a cervical region of a spine); or can be configured for attachment to other regions of the spine (in other words, the thoracic region or lumbar region).

The material 12 of the spinal plate can comprise, consist essentially of, or consist of aromatic polyamide material, either alone, or as a composite with carbon. In some aspects, such aromatic polyamide material can comprise, consist essentially of, or consist of para-aramid, such as, for example, Kevlar™.

If the spinal plate consists of aromatic polyamide material, the plate will be entirely non-metallic, and thus various of the above-discussed prior art problems associated with metallic structures can be avoided. For instance, scattering of diagnostic radiation by the plate can be significantly reduced, or even eliminated, utilizing non-metallic compositions in accordance with aspects of the present invention. Also, utilization of non-metallic compositions can eliminate the problematic triggering of metal detectors that can be associated with the metallic compositions conventionally utilized for the plates.

FIG. 1 shows plate 10 having a maximum width 15, and a maximum length 17; and FIG. 2 shows plate 10 having a maximum thickness 19. The maximum width and maximum length can be conventional. A typical conventional length will be from about 14 mm to about 90 mm, and a typical conventional width will be from about 10 mm to about 20 mm.

The maximum thickness 19 of plate 10 can be significantly thinner than conventional devices due to the strength of the material utilized in the plate, and can, for example, be less than 1.5 mm, less than 1.2 mm, or even less than 1 mm.

The reduced thickness of plate 10 relative to conventional plates can eliminate prior art problems, such as, for example, the problem of patients feeling a cervical plate when they swallow.

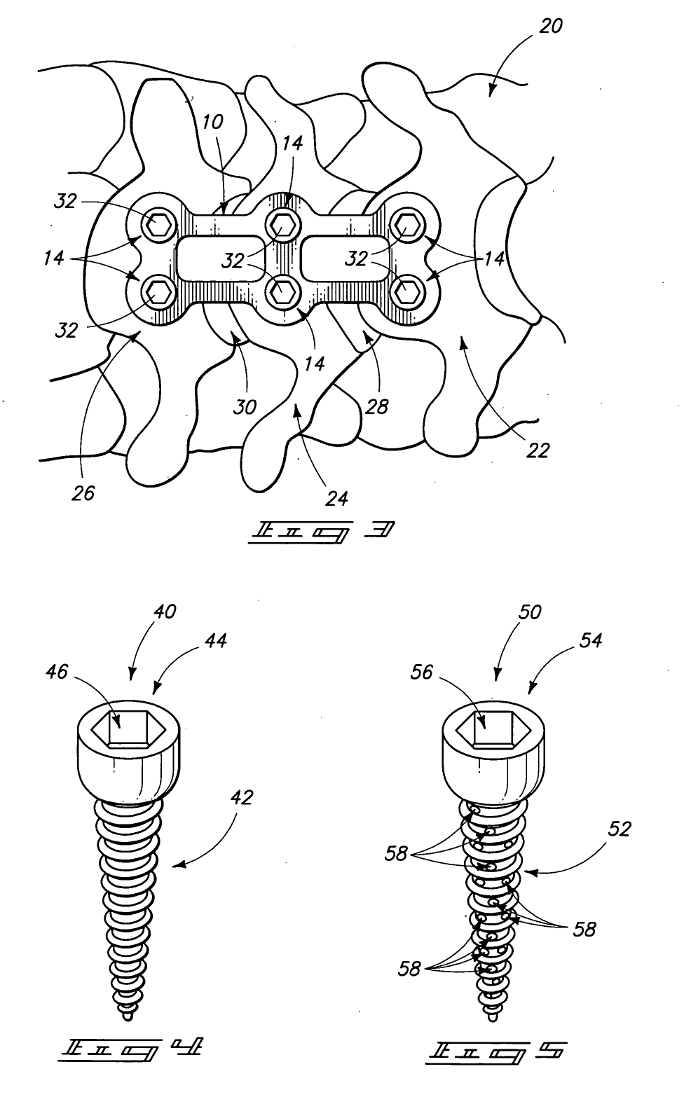

FIG. 3 shows a therapeutic construction comprising plate 10 joined to a region of a spinal column 20.

The spinal column includes a plurality of vertebra 22, 24 and 26, with intervening discs 28 and 30. Typically, a segment of the spinal column is understood to comprise a pair of vertebra and the disc between them; and accordingly the shown portion of the spinal column comprises two segments, with the vertebra 24 shared between the segments. The shown portion of the spinal column can correspond to any region of the spinal column, or in other words can comprise the cervical region, thoracic region or lumbar region of the spinal column.

Plate 10 is shown to extend across the two segments of the spinal column. It is to be understood that the invention also includes spinal plates which extend across only one segment of a spinal column, as well as including plates which extend across more than two segments of a spinal column.

Fasteners 32 are provided within the holes 14 of the spinal plate 10. The fasteners can be any suitable fasteners, including, for example, pins and screws. The fasteners can be conventional fasteners, which are typically metallic. However, in some aspects of the invention it can be preferred that the fasteners be non-metallic to avoid various problems that can be associated with metallic implants (such as those discussed in the “Background” section of this disclosure).

If the fasteners are non-metallic, they can comprise, consist essentially of, or consist of aromatic polyamide material, either alone, or as a composite with carbon. In particular aspects, at least some of the fasteners can comprise, consist essentially of, or consist of para-aramid, either alone, or as a composite with carbon.

In typical aspects of the invention, the fasteners 32 will be screws. Exemplary screws are shown in FIGS. 4 and 5 as screws 40 and 50, respectively. Such screws can be metallic or non-metallic, but for the reasons discussed above it can be advantageous for the screws to be non-metallic. The screw 40 of FIG. 4 comprises a threaded shaft 42 joining to a head 44. The head has a tool-engagement slot 46 extending therein. The tool-engagement slot is configured to receive a tool utilized for screwing the screw 40 into a vertebra, and can correspond to a slot configured to receive any appropriate tool for screwing the screw into bone. The slot can be configured to receive, for example, a Phillips screwdriver or other cross-slotted screwdriver, a straight-slotted screwdriver, an Allen wrench, a Torx wrench, etc. The shown tool-engagement slot is configured for receiving a hexagonal-headed wrench.

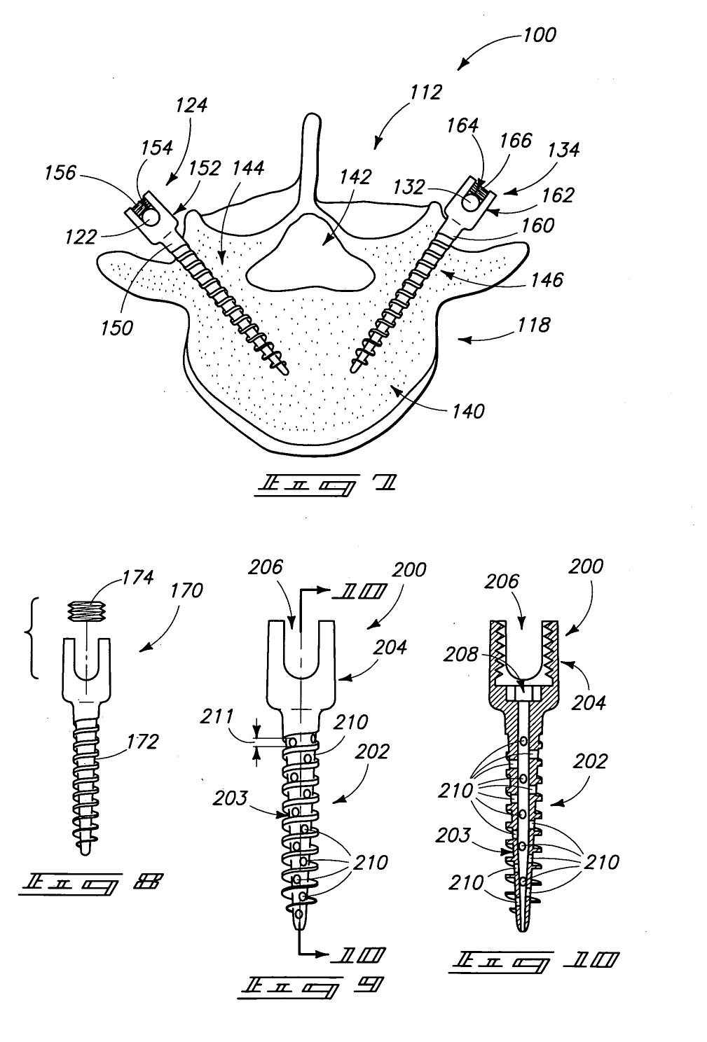

The screw 50 is similar to the screw 40, in that it comprises a threaded shaft 52, a head 50 for joining to the shaft, and a tool-engagement slot 56 extending within the head. However, screw 50 differs from screw 40 in that screw 50 comprises pores 58 extending therein. Such pores can be similar to pores discussed below with reference to FIGS. 9-12, and accordingly can be configured for retaining bone cement and/or bone-growth stimulating material. Additionally, or alternatively, the pores can be configured so that bone can grow into the screw to enhance union of the screw with adjacent skeletal structure. Further, a cannula (not shown in FIG. 5, but discussed below with reference to FIG. 10) can extend longitudinally through screw 50.

A difficulty in attaching implant constructions to skeletal regions is that numerous conditions and diseases can lead to softened or weakened bone structures to which it is difficult to achieve robust union. For instance, osteoporosis increases bone porosity, which leads to softened bone structures. Implant constructions can frequently be screwed to osteoporotic bones in a problem-free manner. However, the screws holding the implant constructions to the bones can subsequently loosen from the bones through the normal forces exerted on the screws and implant constructions during ordinary day-to-day activities, or even can be pulled out of the bones if large forces occur.

Similar difficulties to those confronted with softened or weakened bone structures can also occur with normal, healthy bone structures.

In light of the problems confronted in obtaining and maintaining robust union of screws with bones, it can be preferred to utilize porous screws of the type shown in FIG. 5, instead of non-porous screws of the type shown in FIG. 4.

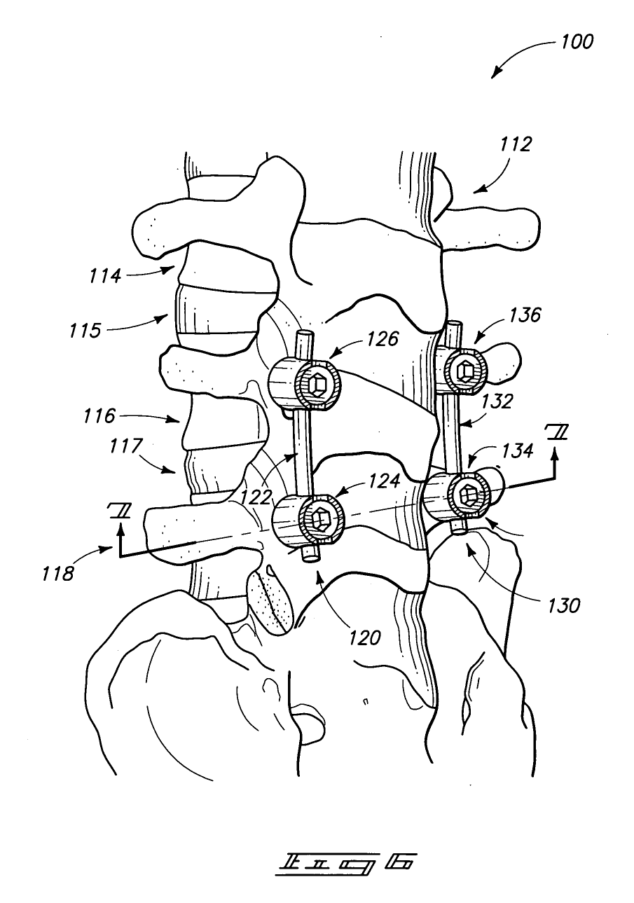

The plates discussed above are but one type of implant construction that can be attached to a skeletal region with screws. An exemplary procedure of utilizing screws to attach another type implant construction to a skeletal region is described with reference to FIGS. 6-8.

Referring to FIG. 6, such shows an assembly 100 comprising a spine 112 and a pair of implant constructions 120 and 130.

The spine comprises a series of vertebrae 114, 116 and 118 separated by disks 115 and 117.

The implant construction 120 comprises a rod 122 held between a pair of support structures 124 and 126; and the implant construction 130 comprises a rod 132 held between a pair of support structures 134 and 136. The rods 122 and 132 would traditionally be relatively rigid metal bars (such as, for example, titanium bars), but it is becoming increasingly common to utilize somewhat flexible materials (such as, for example, polymeric materials) for the rods to provide increased mobility. In some aspects of the invention, the rods can comprise, consist essentially of or consist of aromatic polyamide material, either alone, or as a composite with carbon.

The support structures 124, 126, 134 and 136 contain screws inserted into the pedicles of the vertebra. In some aspects of the present invention, such screws can be non-metallic. The screws can, for example, comprise, consist essentially of, or consist of aromatic polyamide material, either alone, or as a composite with carbon.

The screws have heads configured to enable retention of the rods. The support structures also comprise plugs inserted into the heads of the screws to lock the rods into the screws, as described in more detail below with reference to FIGS. 7 and 8. In some aspects, the plugs can be non-metallic, and can, for example, comprise, consist essentially of, or consist of aromatic polyamide material, either alone, or as a composite with carbon.

As mentioned above, a spinal segment is typically defined as a disc and the pair of vertebrae on opposing sides of the disc. Thus, the implant constructions 120 and 130 can each be considered to comprise a pair of pedicle screws on opposing sides of a spinal segment, and a rod joining the pedicle screws to one another.

FIG. 7 shows a cross-section through vertebra 118, and through support structures 124 and 134 of the constructions 120 and 130. The cross-section of FIG. 7 shows various anatomical features of vertebra 118, including the vertebral body 140, spinal canal 142 (through which the spinal nerve (not shown) passes), and pedicles 144 and 146. The cross-section of FIG. 7 also shows that support structures 124 and 134 comprise pedicle screws 150 and 160, respectively, which extend through pedicles 144 and 146, and into the vertebral body 140.

The pedicle screws 150 and 160 have heads 152 and 162, respectively. Such heads have channels 154 and 164 extending therein. The channels are configured to receive rods 122 and 132, and are further configured to receive plugs (or caps) 156 and 166 which retain the rods within the channels. The particular shown screws have threads within the channels. The threads within the channels receive threads of the plugs so that the plugs can be threadedly engaged within the channels to retain the rods. However, as will be recognized by persons of ordinary skill in the art, there are numerous other structural designs for pedicle screw heads which can be utilized for retaining rods to the pedicle screws. Also, persons of ordinary skill in the art will recognize that pedicle screws can be utilized for retaining other implant structures besides rods.

FIG. 8 shows a disassembled structure 170 comprising a pedicle screw 172 and a cap (or plug) 174. The screw 172 is identical to the screws 150 and 160 discussed above the reference to FIG. 7, and the cap 174 is identical to the caps 156 and 166. The disassembled structure of FIG. 8 shows that the cap is configured to threadedly engage within the channel in the head of screw 172.

As mentioned above, the invention includes aspects in which one or more pores are incorporated within screws. Such pores can be configured so that bone structure grows into the pores to improve the union of the screws with bone. The bone structure growth into the pores can be enhanced by providing one or more bone-growth-stimulating compositions within the pores.

FIGS. 9 and 10 show an exemplary screw 200 illustrating an aspect in which pores are provided within the screw. The screw 200 is similar to the screws 150, 160 and 170 discussed above with reference to FIGS. 6-8. Accordingly, screw 200 comprises a threaded shaft 202 and a head 204 joined to the shaft. The shaft 202 is shown to be fully threaded, but it is to be understood that the shaft could also be only partially threaded in some applications. The screw 200 can be non-metallic; and can, for example, comprise, consist essentially of, or consist of aromatic polyamide material, either alone, or as a composite with carbon.

The head 204 has a channel 206 extending therein. Such channel is threaded, as is apparent from the cross-sectional view of FIG. 10. The channel is configured so that a cap (or plug) can be utilized for retaining a rod within the channel. The screw 200 of FIGS. 9 and 10 can have any suitable dimensions of length and diameter.

The screw 200 of FIGS. 9 and 10 comprises a longitudinally-extending opening (also referred to herein as a cannula) 208 within the shaft, and a plurality of pores 210 (only some of which are labeled) extending through the shaft and to the opening 208. In some aspects, the shaft 202 can be considered to comprise a lateral sidewall 203, and the pores can be considered to extend through such lateral sidewall to the longitudinally-elongated opening 208. The pores and opening are configured to enable bone growth to extend into the screw 200.

Persons of ordinary skill in the art will recognize that a tool can be readily configured for inserting screw 200 into a bone.

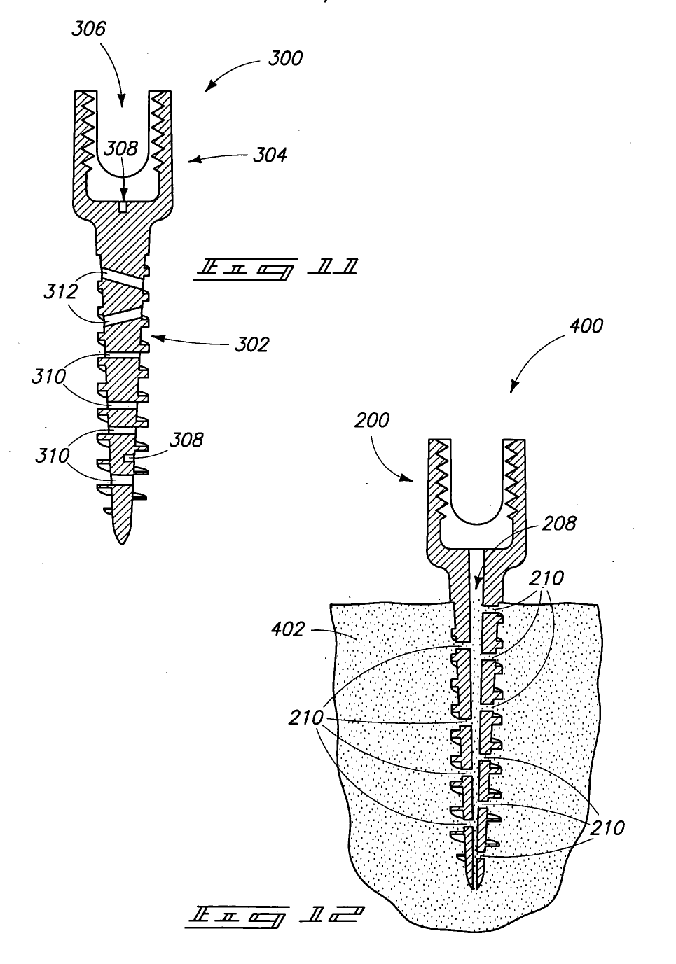

The size of the longitudinally-elongated opening, size of the pores, and number of pores can vary depending on the intended application of screw 200. In some applications (discussed below with reference to FIG. 11), the longitudinally-elongated opening can be omitted. In some aspects of such applications, at least some of the pores can extend entirely through the screw (i.e., entirely from one lateral side of the screw to the opposing lateral side of the screw).

In applications in which the longitudinally-elongated opening is provided, the longitudinally-elongated opening can have any suitable length relative to the length of the shaft. In the shown application, the longitudinally-elongated opening is about the same length as the length of the shaft, but in other applications the longitudinally-elongated opening can be substantially shorter than the overall length of the shaft. Typically, however, if the longitudinally-elongated opening is provided within the shaft, the longitudinally-elongated opening will be at least about one third of the length of the shaft. The longitudinally-elongated opening can function to enable bone growth to extend within the screw, and in some applications (discussed below) the longitudinally-elongated opening can also be utilized for provision of bone-growth-stimulating compositions and/or bone cement. Alternatively, or additionally, the longitudinally-elongated opening can be utilized as a reservoir for retaining bone-growth-stimulating compositions and/or bone cement. In some aspects of the invention, it can be preferred that the longitudinally-elongated opening extend to the channel in the head, as shown, to enable bone-growth-stimulating compositions and/or bone cement to be injected into the longitudinally-elongated opening after the screw is at least partially inserted into a bone.

Regardless of whether or not a longitudinally-elongated opening is provided within the screw 200, there will be at least one pore (or cavity) extending into or through the wall of the shaft, and specifically through the bottom (i.e., tip) of the shaft and/or through a sidewall of the shaft. In the shown aspect of the invention, a pore extends through the bottom of the shaft, and several pores extend through the sidewall of the shaft. If the shaft is only partially threaded, one or more pores can extend into non-threaded portions of the shaft in addition to, or alternatively to, having one or more pores extending into threaded portions of the shaft.

Pores 210 can have any suitable size for enabling sufficient bone growth to occur within the pores to assist in retaining the screw to a bone. The shown pores are approximately circular along a lateral cross-section, with an exemplary pore having a cross-sectional diameter 211 of, for example, from about 0.1 mm to about 3 mm. The pores can extend through the sidewall 203 at any suitable angle. In some aspects, the pores will extend substantially orthogonally to a normal (i.e., longitudinal) axis of the screw, and in other applications at least some of the pores will extend at an angle which is not substantially orthogonal to the normal axis of the screw.

Although the screw of FIG. 10 is shown as a pedicle screw, it is to be understood that the screw can alternatively be another type of screw suitable for engaging bone. For instance, the screw can be a cervical screw, or a screw suitable for engaging regions other than the spine, including, for example, screws suitable for retaining hip implants, knee implants or shoulder implants; screws suitable for being utilized alone to retain bone fragments; screws suitable for retaining various plates, cages and rods; or any other screws utilized for reconstruction, repair and/or support of skeletal regions. Any of such screws can be non-metallic; and can, for example, comprise, consist essentially of, or consist of aromatic polyamide material, either alone, or as a composite with carbon.

FIG. 11 shows a screw 300 similar to the screw 200 discussed above with reference to FIG. 10, but lacking a longitudinally-extending cannula. Screw 300 comprises a threaded shaft 302 and a head 304 joined to the shaft. Screw 300 further comprises a threaded channel 306 extending into the head. Channel 306 has a slot 308 therein for receiving a tool which can be utilized for screwing the screw 300 into a bone. Such slot can be part of receptacle suitable for receiving, for example, a Phillips screwdriver or other cross-slotted screwdriver, a straight-slotted screwdriver, an Allen wrench, a Torx wrench, etc.

Screw 300 comprises pores 308, 310 and 312 analogous to the pores 210 associated with the screw 200 of FIGS. 9 and 10, with such pores being laterally-elongated openings in the embodiment of FIG. 11. The pores 308, 310 and 312 are configured for receiving bone structure grown into the pores. In the shown aspect, some of the pores extend entirely through the screw (specifically, pores 310 and 312) while one of the pores only extends partially into the screw (specifically, pore 308). Also, some of the pores are shown extending along approximately horizontal axes relative to a vertical axis defined by a normal axis of the screw (specifically, pores 310) while some of the pores are shown extending along axes tipped relative to such horizontal axes (specifically, pores 312).

Regardless of whether a porous screw is configured With a longitudinally-extending opening of the type shown in FIGS. 9 and 10, or without such longitudinally-elongated opening as shown in FIG. 11, bone-growth-stimulating material and/or various cements and bone adhering materials can be provided in one or more of the pores. For instance bone-growth-stimulating material can be provided to enhance growth of bone into the pores and/or polymethyl-methacrylate (PMMA) (a form of bone cement) can be provided within the pores to enhance adhesion to bone. If the longitudinally-elongated opening is present, the bone-growth-stimulating material and/or PMMA can be provided by injection of the bone-growth-stimulating material and/or PMMA through the longitudinally-elongated opening and into the pores joined to the opening before, after, and/or during screwing of the screw into bone. If the longitudinally-elongated opening is not present, the bone-growth-stimulating material and/or PMMA will typically be provided in the pores prior to screwing of the screw into the bone. Also, even if the longitudinally-elongated opening is present, the bone-growth-stimulating material and/or PMMA can be provided within the pores but not within the longitudinally-elongated opening, or vice versa. Further, if the longitudinally-elongated opening is present but some of the pores do not join with the opening, bone-growth-stimulating material and/or PMMA can be provided within the pores that do not join with the opening prior to screwing of the screw into the bone.

The bone-growth-stimulating material can comprise any composition or combination of compositions which stimulate bone growth. For instance, the bone-growth-stimulating material can comprise one or both of fibronectin and hydroxyapatite. Additionally, or alternatively, the bone-growth-stimulating material can comprise one or more bone morphogenetic proteins (bmp's) such as, for example, bmp2 and/or bmp7; and/or other osteo-inductive conductors. In some aspects, at least portions of the outer sidewall surfaces of the screw shafts (and particularly at least portions of the threaded surfaces of the shafts) are coated with one or both of fibronectin and hydroxyapatite to enhance union of the screws to bone. Such coating can be utilized in addition to the provision of bone-growth-stimulating material and/or bone cement in the pores and/or cannula of porous screws.

FIG. 12 shows an assembly 400 comprising the screw 200 (described above with reference to FIGS. 9 and 10) embedded in a bone 402. Structure, or matrix, of the bone is shown extending into the pores 210 of the screw, and also into the longitudinally-elongated opening 208. The bone structure within the pores and longitudinally-elongated opening enhances union of the screw with the bone. Such can alleviate prior art problems of screw loosening and screw pullout that could otherwise occur. Advantages of having bone growing into pores associated with a screw can occur in numerous applications, but can be particularly significant for patients suffering from bone-weakening ailments such as, for example, osteopenia or osteoporosis.

The shown screw 200 is a pedicle screw, and in the diagram of FIG. 12 bone has grown into the pores of the screw prior to assembly of the spine-stabilizing implant that is ultimately to be retained by the screw (specifically, prior to provision of rods and plugs of the type described with reference to FIG. 6). This can be a preferred aspect of the invention. Specifically, a porous screw can be fastened to a bone, and then left attached to the bone for a period of time sufficient to have bone growth extend into pores of the screw prior to attachment of an implant construction to the screw. This can enable the screw to become tightly joined with the bone through the growth of bone structure into the pores associated with the screw prior to providing stresses on the screw associated with an attached implant construction.

In the case of pedicle screws, for example, significant stresses can be applied to the screws once that rods are tightly joined to the screws. Such stresses can cause the screws to pull out of the pedicles if the stresses occur before a strong union of the screws with the pedicles has been achieved. Accordingly, it can be advantageous to wait until bone matrix material has grown into the pores of the pedicle screws (and in some aspects adhered to a surface of the screw) before tightly attaching the rods to the pedicle screws. Similar considerations can occur with screws other than pedicle screws in other applications in which the screws are utilized to support an implant construction, including, for example, applications in which the screws hold cages, plates, shafts and/or rods.

Various of the aspects discussed above for screws can also be applied to vertebral hooks. For instance, vertebral hooks can be formed to be porous; and /or to comprise, consist essentially of, or consist of aromatic polyamide material, either alone, or as a composite with carbon.

Exemplary vertebral hooks are shown in FIGS. 13-16 as hooks 500, 502, 504 and 506, respectively. The hook 506 is shown to have perforated regions (similar to the regions described previously relative the screws). The perforated regions can be utilized for enhancing union of the hook to a skeletal region, in a manner similar to that discussed above relative to the screws.

The specific aspects of the invention shown in the drawings and described above are but some exemplary aspects of the present invention. It is to be understood that the invention can also include other skeletal support structures comprising, consisting essentially of, or consisting of non-metallic materials; with exemplary non-metallic materials being aromatic polyamide materials, and composites of carbon with aromatic polyamide materials. For instance, various non-metallic materials and composites described herein can be utilized in rods, hooks, screws, vertebral spacers, vertebral replacement structures, and any other implant in which a material having high strength to weight ratio is desired. It is also to be understood that the invention can include other porous structures besides those specifically shown in the drawings and described above.

In compliance with the statute, the invention has been described in language more or less specific as to structural and methodical features. It is to be understood, however, that the invention is not limited to the specific features shown and described, since the means herein disclosed comprise preferred forms of putting the invention into effect. The invention is, therefore, claimed in any of its forms or modifications within the proper scope of the appended claims appropriately interpreted in accordance with the doctrine of equivalents.

The invention claimed is:

1. A spinal plate comprising aromatic polyamide material.

2. The plate of claim 1 comprising a composite of the aromatic polyamide material and carbon.

3. The plate of claim 1 wherein the aromatic polyamide material comprises para-aramid.

4. The plate of claim 1 wherein the aromatic polyamide material is para-aramid; and wherein the plate consists of the para-aramid.

5. The plate of claim 1 being entirely non-metallic.

6. The plate of claim 1 wherein the aromatic polyamide material is para-aramid; and wherein the plate consists of a composite of the para-aramid and carbon.

7. The plate of claim 1 being a cervical plate.

8. The plate of claim 7 being less than 1.5 millimeters thick.

9. The plate of claim 7 being less than 1.2 millimeters thick.

10. The plate of claim 7 being less than 1 millimeter thick.

11. A cervical plate that is less than or equal to about 1.5 millimeters thick, and that comprises a composite of aromatic polyamide material and carbon.

12. The plate of claim 11 being entirely non-metallic.

13. The plate of claim 11 consisting of the composite.

14. The plate of claim 11 being less than 1.2 millimeters thick.

15. The plate of claim 11 being less than 1 millimeter thick.

16. The plate of claim 11 wherein the aromatic polyamide material comprises para-aramid.

17. The plate of claim 11 wherein the aromatic polyamide material consists of para-aramid.

18. A vertebral hook comprising one or more pores extending therein, with said one or pores being configured to receive bone structure grown from the bone adjacent the hook to enhance union of the hook with the bone.

19. The hook of claim 18 consisting of a composite of carbon and aromatic polyamide material.

20. The hook of claim 18 having bone cement within at least one of said one or more pores.

21. The hook of claim 18 having bone-growth-stimulating material within at least one of said one or more pores.

22. The hook of claim 21 wherein the bone-growth-stimulating material comprises fibronectin and hydroxyapatite.

23. A non-metallic vertebral hook comprising aromatic polyamide material.

24. The hook of claim 23 comprising a composite of the aromatic polyamide material and carbon.

25. The hook of claim 23 wherein the aromatic polyamide material comprises para-aramid.

26. The hook of claim 23 wherein the aromatic polyamide material is para-aramid; and wherein the plate consists of the para-aramid.

27. The hook of claim 23 wherein the aromatic polyamide material is para-aramid; and wherein the plate consists of a composite of the para-aramid and carbon.

28. The hook of claim 23 having one or more pores extending therein.

29. A therapeutic construction, comprising:
a segment of a spinal column comprising a pair of vertebrae and a disk between the vertebrae; and
a non-metallic structure attached to each vertebra of the pair of vertebrae with non-metallic fasteners.

30. The construction of claim 29 wherein the non-metallic structure is a plate.

31. The construction of claim 30 wherein the plate comprises aromatic polyamide material.

32. The construction of claim 30 wherein the plate comprises a composite of aromatic polyamide material and carbon.

33. The construction of claim 29 wherein the non-metallic structure is a rod.

34. The construction of claim 33 wherein the rod comprises aromatic polyamide material.

35. The construction of claim 33 wherein the rod comprises a composite of aromatic polyamide material and carbon.

36. The construction of claim 29 wherein the segment is within a cervical region of the spinal column.

37. The construction of claim 29 wherein the segment is within a thoracic region of the spinal column.

38. The construction of claim 29 wherein the segment is within a lumbar region of the spinal column.

39. The construction of claim 29 wherein at least one of said fasteners has one or more pores extending therein; and wherein bone grown from at least one of the vertebrae is within at least one of the one or more pores.

40. The construction of claim 29 wherein at least one of fasteners is a vertebral hook.

41. The construction of claim 40 wherein the hook comprises aromatic polyamide material.

42. The construction of claim 40 wherein the hook comprises a composite of aromatic polyamide material and carbon.

43. The construction of claim 29 wherein at least one of the fasteners is a screw.

44. The construction of claim 43 wherein the screw comprises aromatic polyamide material.

45. The construction of claim 44 wherein the aromatic polyamide material comprises para-aramid.

46. The construction of claim 43 wherein the screw consists of aromatic polyamide material.

47. The construction of claim 46 wherein the aromatic polyamide material comprises para-aramid.

48. The construction of claim 43 wherein the screw consists of a composite of aromatic polyamide material and carbon.

49. The construction of claim 48 wherein the aromatic polyamide material comprises para-aramid.

50. The construction of claim 48 wherein the screw has one or more pores extending therein; and wherein bone grown from at least one of the vertebrae is within at least one of the one or more pores.

51. The construction of claim 48 wherein all of the fasteners are screws consisting of the composite of aromatic polyamide material and carbon, have one or more pores extending therein, and have bone from at least one of the vertebrae within at least one pore.

52. A screw configured to directly engage a bone, the screw comprising:
a shaft that is at least partially threaded;
at least one pore extending into the shaft and configured to receive bone structure grown from the bone to enhance union of the screw with the bone; and
wherein the screw comprises aromatic polyamide material.

53. The screw of claim 52 wherein the aromatic polyamide material comprises para-aramid.

54. The screw of claim 52 comprising a composite of carbon and the aromatic polyamide material.

55. The screw of claim 52 wherein the screw is entirely non-metallic.

56. The screw of claim 52 wherein the aromatic polyamide material is para-aramid, and wherein the screw consists of a composite of carbon and the para-aramid.

57. The screw of claim 56 being a cervical screw.

58. The screw of claim 56 being a pedicle screw.

59. The pedicle screw of claim 58 having a length of the shaft, and having a longitudinally-elongated opening within the shaft which extends along at least about one-third of the length of the shaft; the pedicle screw further having a lateral sidewall along the shaft, and having one or more pores extending through the lateral sidewall and to the longitudinally-elongated opening.

60. The pedicle screw of claim 59 comprising at least two pores extending through the lateral sidewall and to the longitudinally-elongated opening.

61. The pedicle screw of claim 59 having bone cement within at least one of said one or more pores.

62. The pedicle screw of claim 59 having bone-growth-stimulating material within at least one of said one or more pores.

63. The pedicle screw of claim 62 wherein the bone-growth-stimulating material comprises fibronectin and hydroxyapatite.

64. The pedicle screw of claim 59 having a head attached to the shaft, with said head having a channel extending therein; and wherein the longitudinally-elongated opening extends into the shaft from the channel in the head.
